(19) United States
(12) Patent Application Publication
(10) Pub. No.: US 2010/0022571 A1
(43) Pub. Date: Jan. 28, 2010

(54) SUBSTITUTED PYRAZOLO [3,4-B]PYRIDINES AS PHOSPHODIESTERASE INHIBITORS

(76) Inventors: Venkata P. Palle, Pune (IN); Sarala Balachandran, Mumbai (IN); Nidhi Gupta, New Delhi (IN); Vinayak Vasantrao Khairnar, Nashik (IN); Mandadapu Raghuramaiah, Guntur (IN); Abhijit Ray, New Delhi (IN); Sunanda Ghose Dastidar, New Delhi (IN)

Correspondence Address: Ranbaxy Inc., Intellectual Property Department, 600 College Road East, PRINCETON, NJ, 08540, US

(21) Appl. No.: 12/066,914

(22) PCT Filed: Sep. 18, 2006
(86) PCT No.: PCT/IB2006/053349

§ 371 Date: Sep. 3, 2008

(30) Foreign Application Priority Data
Sep. 16, 2005 (IN) 2521/DEL/2005

(51) Int. Cl. A61K 31/437; C07D 471/04; A61P 29/00; A61P 25/00; A61P 11/00; A61P 1/00

(52) U.S. Cl. 514/278; 546/15

(57) ABSTRACT

The present invention relates to phosphodiesterase (PDE) type IV selective inhibitors. Processes for the preparation of disclosed compounds, pharmaceutical compositions containing the disclosed compounds and their use as PDE type IV selective inhibitors are provided. Prepared compounds correspond to structure XIV.

### FIELD OF THE INVENTION

The present invention relates to phosphodiesterase (PDE) type IV selective inhibitors.

Compounds disclosed herein can be useful in the treatment of CNS diseases, AIDS, asthma, arthritis, bronchitis, chronic obstructive pulmonary disease (COPD), psoriasis, allergic rhinitis, shock, atopic dermatitis, Crohn's disease, adult respiratory distress syndrome (ARDS), eosinophilic granuloma, allergic conjunctivitis, osteoarthritis, ulcerative colitis and other inflammatory diseases especially in humans.

Processes for the preparation of disclosed compounds, pharmaceutical compositions containing the disclosed compounds and their use as PDE type IV selective inhibitors are provided.

### BACKGROUND OF THE INVENTION

It is known that cyclic adenosine-3′,5′-monophosphate (cAMP) exhibits an important role of acting as an intracellular secondary messenger (Sutherland et al., Pharmacol. Rev., (1960), 12, 265). Its intracellular hydrolysis to adenosine 5′-monophosphate (AMP) causes number of inflammatory conditions which are not limited to psoriasis, allergic rhinitis, shock, atopic dermatitis, Crohn's disease, adult respiratory distress syndrome (ARDS), eosinophilic granuloma, allergic conjunctivitis, osteoarthritis, ulcerative colitis. PDE4 inhibitors are designed to inhibit the activity of PDE4, the enzyme which breaks down neuronal cAMP. Studies have shown that administering PDE4 inhibitors can have a restorative effect on memory loss in animal models, including those of Alzheimer's disease (Expert Opin. Ther. Targets, (2005) 9(6):1283-1305; Drug Discovery Today, 10(22), (2005). The most important role in the control of cAMP (as well as of cGMP) level is played by cyclic nucleotide phosphodiesterases (PDE) which represent a biochemically and functionally highly variable super family of enzymes. Eleven distinct families of cyclic nucleotide phosphodiesterases with more than 25 gene products are currently recognized. Although PDE I, PDE II, PDE III, PDE IV, and PDE VII all use cAMP as a substrate, only PDE IV and PDE VII are highly selective for hydrolysis of cAMP. Inhibitors of PDE, particularly the PDE IV inhibitors, such as rolipram or Ro-1724 are therefore known as cAMP-enhancers. Immune cells contain type IV and type III PDE, the PDE IV type being prevalent in human mononuclear cells. Thus the inhibition of phosphodiesterase type IV has been a target for modulation and, accordingly, for therapeutic intervention in a range of disease processes.

The initial observation that xanthine derivatives, theophylline and caffeine inhibit the hydrolysis of cAMP led to the discovery of the required hydrolytic activity in the cyclic nucleotide phosphodiesterase (PDE) enzymes. Distinct classes of PDE's have been recognized (Bervo et al., TIPS, (1990), 11, 150), and their selective inhibition has led to improved drug therapy (Nicholus et al., TIPS, (1991), 12, 19). Thus it was recognized that inhibition of PDE IV could lead to inhibition of inflammatory mediator release (Verghese et al., J. Mol. Cell. Cardiol., (1989), 12 (Suppl. II), S 61) and airway smooth muscle relaxation.

WO 2003/047520 discloses substituted aminomethyl compounds and derivatives thereof, which have been described to be useful as inhibitors of factor Xa. WO 2000/59902 discloses aryl sulfonyls, which have been described to be useful as inhibitors of factor Xa. WO 97/48697 discloses substituted azabicyclic compounds and their use as inhibitors of the production of TNF and cyclic AMP phosphodiesterase. WO 98/57951 and U.S. Pat. No. 6,339,099 describe nitrogen containing heteroaromatics and derivatives, which have been said to be the inhibitors of factor Xa.

### SUMMARY OF THE INVENTION

The present invention provides phosphodiesterase (PDE) type IV selective inhibitors, which can be used for the treatment of CNS diseases, AIDS, asthma, arthritis, bronchitis, chronic obstructive pulmonary disease (COPD), psoriasis, allergic rhinitis, shock, atopic dermatitis, Crohn's disease, adult respiratory distress syndrome (ARDS), eosinophilic granuloma, allergic conjunctivitis, osteoarthritis, ulcerative colitis and other inflammatory diseases and the processes for the synthesis of these compounds.

Pharmaceutically acceptable salts, pharmaceutically acceptable solvates, stereoisomers, tautomers, racemates, regioisomers, prodrugs, metabolites, polymorphs or N-oxides of these compounds having the same type of activity are also provided.

Pharmaceutical compositions containing the compounds, which may also contain pharmaceutically acceptable carriers or diluents, can be used for the treatment of CNS diseases, AIDS, asthma, arthritis, bronchitis, chronic obstructive pulmonary disease (COPD), psoriasis, allergic rhinitis, shock, atopic dermatitis, Crohn's disease, adult respiratory distress syndrome (ARDS), eosinophilic granuloma, allergic conjunctivitis, osteoarthritis, ulcerative colitis and other inflammatory diseases.

Other aspects will be set forth in the accompanying description which follows and in part will be apparent from the description or may be learnt by the practice of the invention.

In accordance with one aspect, there are provided compounds having the structure of Formula I:

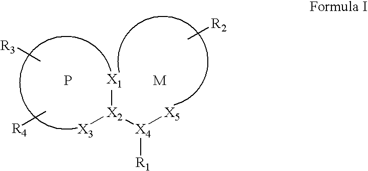

and their pharmaceutically acceptable salts, pharmaceutically acceptable solvates, stereoisomers, tautomers, racemates, regioisomers, prodrugs, metabolites, polymorphs or N-oxides, wherein ring P including X₁, X₂ and X₃ can be a six-membered ring containing 1-3 double bonds wherein X₁ and X₂ can be carbon and X₃ can be nitrogen;
ring M including X₁, X₂, X₄ and X₅ can be a five-membered ring containing 1-2 double bonds wherein X₁ and X₂ can be carbon and X₄ and X₅ can be nitrogen;
R₁ can be hydrogen, alkyl, alkenyl, alkynyl, cycloalkyl, aryl, aralkyl, aralkenyl, (cycloalkyl) alkyl, heterocyclyl, heteroaryl, (heterocyclyl) alkyl or (heteroaryl) alkyl;
R₂ can be hydrogen, alkyl, halogen, cyano, nitro, —SR, —NRR, —(CH₂)ₙOR (wherein R can be hydrogen, alkyl, alkenyl, alkynyl, cycloalkyl, aryl, heteroaryl or heterocyclyl and n can be an integer from 0-2), alkenyl, alkynyl, cycloalkyl, (cycloalkyl) alkyl, aryl, aralkyl, aralkenyl, heterocyclyl, heteroaryl, (heterocyclyl) alkyl or (heteroaryl) alkyl;
R₃ can be —NR₅R₆ (wherein R₅ and R₆ independently can be hydrogen, alkyl, alkenyl, alkynyl, acyl, cycloalkyl, aryl, aralkenyl, aralkyl, (cycloalkyl) alkyl, heterocyclyl, heteroaryl, (heterocyclyl) alkyl or (heteroaryl) alkyl); and
R₄ can be a radical of Formula I a or I b

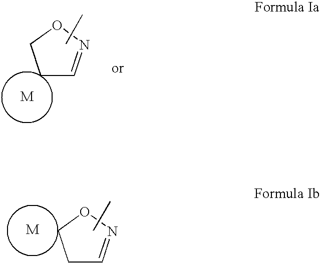

wherein M can be a 3- to 7-membered saturated, partially saturated or unsaturated ring containing carbon atoms wherein one or more carbon atoms can be replaced by heteroatoms selected from O, S(O)ₙ {wherein n can be an integer from 0-2} or —NR-{wherein R is the same as defined earlier}.
The following definitions apply to terms as used herein.

The term “alkyl,” unless otherwise specified, refers to a monoradical branched or unbranched saturated hydrocarbon chain having from 1 to 20 carbon atoms. Alkyl groups can be optionally interrupted by atom(s) or group(s) independently selected from oxygen, sulfur, a phenylene, sulphinyl, sulphonyl group or —NR_{α}—, wherein R_{α} can be hydrogen, alkyl, cycloalkyl, alkenyl, cycloalkenyl, alkynyl, aryl, acyl, aralkyl, —C(═O)OR_{λ}, SOₘR_{ψ} or —C(═O)NR_{λ}R_{π}. This term can be exemplified by groups such as methyl, ethyl, n-propyl, iso-propyl, n-butyl, iso-butyl, sec-butyl, t-butyl, n-pentyl, isopentyl, neopentyl, n-hexyl, n-decyl, tetradecyl, and the like. Alkyl groups may be substituted further with one or more substituents selected from alkenyl, alkynyl, alkoxy, cycloalkyl, cycloalkenyl, acyl, acylamino, acyloxy, alkoxycarbonylamino, azido, cyano, halogen, hydroxy, keto, oxo, thiocarbonyl, carboxy, carboxyalkyl, aryl, heterocyclyl, heteroaryl, (heterocyclyl)alkyl, cycloalkoxy, —CH═N—O(C₁₋₆alkyl), —CH═N—NH(C₁₋₆alkyl), —CH═N—NH(C₁₋₆alkyl)-C₁₋₆alkyl, arylthio, thiol, alkylthio, aryloxy, nitro, aminosulfonyl, aminocarbonylamino, —NHC(═O)R_{λ}, NR_{λ}R_{π}, —C(═O)NR_{λ}R_{π}, —NHC(═O)NR_{λ}R_{π}, —C(═O)heteroaryl, C(═O)heterocyclyl, —O—C(═O)NR_{λ}R_{π}, {wherein R_{λ} and R_{π} are independently selected from hydrogen, halogen, hydroxy, alkyl, alkenyl, alkynyl, alkenyl, alkoxy, cycloalkyl, cycloalkenyl, aryl, aralkyl, heterocyclyl, heteroaryl, heterocyclylalkyl, heteroarylalkyl or carboxy}, nitro or —SOₘR_{ψ} (wherein m is an integer from 0-2 and R_{ψ} is hydrogen, alkyl, alkenyl, alkynyl, cycloalkyl, aralkyl, aryl, heterocyclyl, heteroaryl, heteroarylalkyl or heterocyclylalkyl). Unless otherwise constrained by the definition, alkyl substituents may be further substituted by 1-3 substituents selected from alkyl, alkenyl, alkynyl, carboxy, —

NR_{λ}R_{π}, —C(═O)NR_{λ}R_{π}, —OC(═O)NR_{λ}R_{π}, —NHC(═O)NR_{λ}R_{π}, hydroxy, alkoxy, halogen, CF₃, cyano, and —SOₘR_{ψ}; or an alkyl group also may be interrupted by 1-5 atoms of groups independently selected from oxygen, sulfur or —NR_{α}— (wherein R_{α}, R_{λ}, R_{π}, m and R_{ψ} are the same as defined earlier). Unless otherwise constrained by the definition, all substituents may be substituted further by 1-3 substituents selected from alkyl, alkenyl, alkynyl, carboxy, carboxyalkyl, —NR_{λ}R_{π}, —C(═O)NR_{λ}R_{π}, —O—C(═O)NR_{λ}R_{π}, hydroxy, alkoxy, halogen, CF₃, cyano, and —SOARS (wherein R_{λ}, R_{π}, m and R_{ψ} are the same as defined earlier); or an alkyl group as defined above that has both substituents as defined above and is also interrupted by 1-5 atoms or groups as defined above.

The term “alkenyl,” unless otherwise specified, refers to a monoradical of a branched or unbranched unsaturated hydrocarbon group having from 2 to 20 carbon atoms with cis, trans or geminal geometry. Alkenyl groups can be optionally interrupted by atom(s) or group(s) independently chosen from oxygen, sulfur, phenylene, sulphinyl, sulphonyl and —NR_{α}— (wherein R_{α} is the same as defined earlier). In the event that alkenyl is attached to a heteroatom, the double bond cannot be alpha to the heteroatom. Alkenyl groups may be substituted further with one or more substituents selected from alkyl, alkenyl, alkynyl, alkoxy, cycloalkyl, cycloalkenyl, acyl, acylamino, acyloxy, —NHC(═O)R_{λ}, —NR_{λ}R_{π}, —C(═O)NR_{λ}R_{π}, —NHC(═O)NR_{λ}R_{π}, —O—C(═O)NR_{λ}R_{π}, alkoxycarbonylamino, azido, cyano, halogen, hydroxy, oxo, keto, carboxyalkyl, thiocarbonyl, carboxy, arylthio, thiol, alkylthio, aryl, aralkyl, aryloxy, heterocyclyl, heteroaryl, heterocyclyl alkyl, heteroaryl alkyl, aminosulfonyl, aminocarbonylamino, alkoxyamino, hydroxyamino, alkoxyamino, nitro or SOₘR_{ψ} (wherein R_{λ}, R_{π}, m and R_{ψ} are as defined earlier). Unless otherwise constrained by the definition, alkenyl substituents optionally may be substituted further by 1-3 substituents selected from alkyl, alkenyl, alkynyl, carboxy, hydroxy, alkoxy, halogen, —CF₃, cyano, —NR_{λ}R_{π}, —C(═O)NR_{λ}R_{π}, —O—C(═O)NR_{λ}R_{π} and —SOₘR_{ψ} (wherein R_{λ}, R_{π}, m and R_{ψ} are as defined earlier). Groups, such as ethenyl or vinyl (CH═CH₂), 1-propylene or allyl (—CH₂CH═CH₂), iso-propylene (—C(CH₃)═CH₂), bicyclo[2.2.1]heptene, and the like, exemplify this term.

The term “alkynyl,” unless otherwise specified, refers to a monoradical of an unsaturated hydrocarbon, having from 2 to 20 carbon atoms. Alkynyl groups can be optionally interrupted by atom(s) or group(s) independently chosen from oxygen, sulfur, phenylene, sulphinyl, sulphonyl and —NR_{α}— (wherein R_{α} is the same as defined earlier). In the event that alkynyl groups are attached to a heteroatom, the triple bond cannot be alpha to the heteroatom. Alkynyl groups may be substituted further with one or more substituents selected from alkyl, alkenyl, alkoxy, cycloalkyl, cycloalkenyl, acyl, acylamino, acyloxy, alkoxycarbonylamino, azido, cyano, halogen, hydroxy, keto, oxo, thiocarbonyl, carboxy, carboxyalkyl, arylthio, thiol, alkylthio, aryl, aralkyl, aryloxy, aminosulfonyl, aminocarbonylamino, hydroxyamino, alkoxyamino, nitro, heterocyclyl, heteroaryl, heterocyclylalkyl, heteroarylalkyl, —NHC(═O)R_{λ}, —NR_{λ}R_{π}, —NHC(═O)NR_{λ}R_{π}, —C(═O)NR_{λ}R_{π}, —O—C(═O)NR_{λ}R_{π} or —SOₘR_{ψ} (wherein R_{λ}, R_{π}, m and R_{ψ} are the same as defined earlier). Unless otherwise constrained by the definition, alkynyl substituents optionally may be substituted further by 1-3 substituents selected from alkyl, alkenyl, alkynyl, carboxy, carboxyalkyl, hydroxy, alkoxy, halogen, CF₃, —NR_{λ}R_{π}, —C(═O)NR_{λ}R_{π}, —NHC(═O)NR_{λ}R_{π}, —C(═O)NR_{λ}R_{π}, cyano or —SOₘR_{ψ} (wherein R_{λ}, R_{π}, m and R_{ψ} are the same as defined earlier).

The term “cycloalkyl,” unless otherwise specified, refers to cyclic alkyl groups of from 3 to 20 carbon atoms having a single cyclic ring or multiple condensed rings, which may optionally contain one or more olefinic bonds, unless otherwise constrained by the definition. Such cycloalkyl groups can include, for example, single ring structures, including cyclopropyl, cyclobutyl, cyclooctyl, cyclopentenyl, and the like or multiple ring structures, including adamantanyl, and bicyclo[2.2.1]heptane or cyclic alkyl groups to which is fused an aryl group, for example, indane, and the like. Spiro and fused ring structures can also be included. Cycloalkyl groups may be substituted further with one or more substituents selected from alkyl, alkenyl, alkynyl, alkoxy, cycloalkyl, cycloalkenyl, acyl, acylamino, acyloxy, alkoxycarbonylamino, azido, cyano, halogen, hydroxy, oxo, thiocarbonyl, carboxy, carboxyalkyl, arylthio, thiol, alkylthio, aryl, aralkyl, aryloxy, aminosulfonyl, aminocarbonylamino, —NR_{λ}R_{π}, —NHC(═O)NR_{λ}R_{π}, —NHC(═O)R_{λ}, —C(═O)NR_{λ}R_{π}, —O—C(═O)NR_{λ}R_{π}, nitro, heterocyclyl, heteroaryl, heterocyclylalkyl, heteroarylalkyl or SOₘR_{ψ} (wherein R_{λ}, R_{π}, m and R_{ψ} are the same as defined earlier). Unless otherwise constrained by the definition, cycloalkyl substituents optionally may be substituted further by 1-3 substituents selected from alkyl, alkenyl, alkynyl, carboxy, hydroxy, alkoxy, halogen, CF₃, —NR_{λ}R_{π}, —C(═O)NR_{λ}R_{π}, —NHC(═O)NR_{λ}R_{π}, —OC(═O)NR_{λ}R_{π}, cyano or —SOₘR_{ψ} (wherein R_{λ}, R_{π}, m and R_{ψ} are the same as defined earlier). “Cycloalkylalkyl” refers to alkyl-cycloalkyl group linked through alkyl portion, wherein the alkyl and cycloalkyl are the same as defined earlier.

The term “(cycloalkyl) alkyl” refers to alkyl-cycloalkyl group linked through alkyl portion, wherein the alkyl and cycloalkyl are as defined earlier.

The term “alkoxy” denotes the group O-alkyl wherein alkyl is the same as defined above.

The term “aryl,” unless otherwise specified, refers to aromatic system having 6 to 14 carbon atoms, wherein the ring system can be mono-, bi- or tricyclic and are carbocyclic aromatic groups. For example, aryl groups include, but are not limited to, phenyl, biphenyl, anthryl or napthyl ring and the like, optionally substituted with 1 to 3 substituents selected from halogen (e.g., F, Cl, Br, I), hydroxy, alkyl, alkenyl, alkynyl, cycloalkyl, alkoxy, acyl, aryloxy, CF₃, cyano, nitro, COOR_{ψ}, NHC(═O)R_{λ}, —NR_{λ}R_{π}, —C(═O)NR_{λ}R_{π}, —NHC(═O)NR_{λ}R_{π}, —O—C(═O)NR_{λ}R_{π}, —SOₘR_{ψ}, carboxy, heterocyclyl, heteroaryl, heterocyclylalkyl, heteroarylalkyl or amino carbonyl amino, mercapto, haloalkyl, optionally substituted aryl, optionally substituted heterocyclylalkyl, thioalkyl, —CONHR_{π}, —OCOR_{π}, —COR_{π}, —NHSO₂R_{π}, or —SO₂NHR_{π} (wherein R_{λ}, R_{π}, m and R_{ψ} are the same as defined earlier). Aryl groups optionally may be fused with a cycloalkyl group, wherein the cycloalkyl group may optionally contain heteroatoms selected from O, N or S. Groups such as phenyl, naphthyl, anthryl, biphenyl, and the like exemplify this term.

The term “aralkyl,” unless otherwise specified, refers to alkyl-aryl linked through an alkyl portion (wherein alkyl is as defined above) and the alkyl portion contains 1-6 carbon atoms and aryl is as defined below. Examples of aralkyl groups include benzyl, ethylphenyl, propylphenyl, naphthylmethyl and the like.

The term “aralkenyl,” unless otherwise specified, refers to alkenyl-aryl linked through alkenyl (wherein alkenyl is as defined above) portion and the alkenyl portion contains 1 to 6 carbon atoms and aryl is as defined below.

The term “aryloxy” denotes the group O-aryl, wherein aryl is as defined above.

The term “cycloalkoxy” denotes the group O-cycloalkyl, wherein cycloalkyl is as defined above.

The term “carboxy,” as defined herein, refers to —C(═O)OR_{f}, wherein R_{f} is the same as defined above.

The term “heteroaryl,” unless otherwise specified, refers to an aromatic ring structure containing 5 or 6 ring atoms or a bicyclic or tricyclic aromatic group having from 8 to 10 ring atoms, with one or more heteroatom(s) independently selected from N, O or S optionally substituted with 1 to 4 substituent(s) selected from halogen (e.g., F, Cl, Br, I), hydroxy, alkyl, alkenyl, alkynyl, cycloalkyl, acyl, carboxy, aryl, alkoxy, aralkyl, cyano, nitro, heterocyclyl, heteroaryl, —NR_{λ}R_{π}, CH═NOH, —(CH₂)_{w}C(═O)R_{η} {wherein w is an integer from 0-4 and R_{η}, is hydrogen, hydroxy, OR_{λ}, NR_{λ}R_{π}, —NHOR_{ω} or —NHOH}, —C(═O)NR_{λ}R_{π}—NHC(═O)NR_{λ}R_{π}, —SOₘR_{ψ}, —O—C(═O)NR_{λ}R_{π}, —O—C(═O)R_{λ}, or —O—C(═O)OR_{λ} (wherein m, R_{ψ}, R_{λ} and R_{π} are as defined earlier and R_{ω} is alkyl, cycloalkyl, aryl, heteroaryl, heterocyclyl, heteroarylalkyl or heterocyclylalkyl). Unless otherwise constrained by the definition, the substituents are attached to a ring atom, i.e., carbon or heteroatom in the ring. Examples of heteroaryl groups include oxazolyl, imidazolyl, pyrrolyl, 1,2,3-triazolyl, 1,2,4-triazolyl, tetrazolyl, thiazolyl, oxadiazolyl, benzoimidazolyl, thiadiazolyl, pyridinyl, pyridazinyl, pyrimidinyl, pyrazinyl, thienyl, isoxazolyl, triazinyl, furanyl, benzofuranyl, indolyl, benzthiazinyl, benzthiazinonyl, benzoxazinyl, benzoxazinonyl, quinazonyl, carbazolyl phenothiazinyl, phenoxazinyl, benzothiazolyl or benzoxazolyl, and the like.

The term “heterocyclyl,” unless otherwise specified, refers to a non-aromatic monocyclic or bicyclic cycloalkyl group having 5 to 10 atoms wherein 1 to 4 carbon atoms in a ring are replaced by heteroatoms selected from O, S or N, and optionally are benzofused or fused heteroaryl having 5-6 ring members and/or optionally are substituted, wherein the substituents are selected from halogen (e.g., F, Cl, Br, I), hydroxy, alkyl, alkenyl, alkynyl, cycloalkyl, acyl, optionally substituted aryl, alkoxy, alkaryl, cyano, nitro, oxo, carboxy, optionally substituted heterocyclyl, optionally substituted heterocyclylalkyl, optionally substituted heteroaryl, —O—C(═O)R_{λ}, —O—C(═O)OR_{λ}, —C(═O)NR_{λ}R_{π}, SOₘR_{ψ}, —O—C(═O)NR_{λ}R_{π}, —NHC(═O)NR_{λ}R_{π}, —NR_{λ}R_{π}, mercapto, haloalkyl, thioalkyl, —COOR_{ψ}, —COONHR_{λ}, —COR_{λ}, —NHSO₂R_{λ} or SO₂NHR_{λ} (wherein m, R_{ψ}, R_{λ} and R_{π} are as defined earlier) or guanidine. Heterocyclyl can optionally include rings having one or more double bonds. Such ring systems can be mono-, bi- or tricyclic. Carbonyl or sulfonyl group can replace carbon atom(s) of heterocyclyl. Unless otherwise constrained by the definition, the substituents are attached to the ring atom, i.e., carbon or heteroatom in the ring. Also, unless otherwise constrained by the definition, the heterocyclyl ring optionally may contain one or more olefinic bond(s). Examples of heterocyclyl groups include oxazolidinyl, tetrahydrofuranyl, dihydrofuranyl, benzoxazinyl, benzthiazinyl, imidazolyl, benzimidazolyl, tetrazolyl, carbaxolyl, indolyl, phenoxazinyl, phenothiazinyl, dihydropyridinyl, dihydroisoxazolyl, dihydrobenzofuryl, azabicyclohexyl, thiazolidinyl, dihydroindolyl, pyridinyl, isoindole 1,3-dione, piperidinyl, tetrahydropyranyl, piperazinyl, 3H-imidazo[4,5-b]pyridine, isoquinolinyl, 1H-pyrrolo[2,3-b]pyridine or piperazinyl and the like.

“(Heterocyclyl) alkyl” refers to alkyl-heterocyclyl group linked through alkyl portion, wherein the alkyl and heterocyclyl are as defined earlier.

““Acyl” refers to —C(═O)R″ wherein R″ is selected from hydrogen, alkyl, cycloalkyl, aryl, aralkyl, heteroaryl, heterocyclyl, heteroarylalkyl or heterocyclylalkyl.

“Thiocarbonyl” refers to —C(═S)H. “Substituted thiocarbonyl” refers to —C(═S) R′″, wherein R″′ is selected from alkyl, cycloalkyl, aryl, aralkyl, heteroaryl, heterocyclyl, heteroarylalkyl, heterocyclylalkyl, amine or substituted amine. Unless otherwise constrained by the definition, all substituents optionally may be substituted further by 1-3 substituents selected from alkyl, aralkyl, cycloalkyl, aryl, heteroaryl, heterocyclyl, carboxy, hydroxy, alkoxy, halogen, CF₃, cyano, —C(═O)NR_{f}R_{q}, —O(C═O)NR_{f}R_{q} (wherein R_{f} and R_{q} are the same as defined earlier), —(SO)ₙR_{d} (wherein n and R_{d} are the same as defined earlier).

“Amine,” unless otherwise specified, refers to —NH₂. “Substituted amino” unless otherwise specified, refers to a group —N(Rₖ)₂ wherein each Rₖ is independently selected from the group hydrogen provided that both Rₖ groups are not hydrogen (defined as “amino”), alkyl, alkenyl, alkynyl, aralkyl, cycloalkyl, aryl, heteroaryl, heterocyclyl, heterocyclylalkyl, heteroarylalkyl, acyl, S(O)ₘR_{ψ} (wherein m and R_{ψ} are the same as defined above), —C(═Rᵥ)NR_{λ}R_{y} (wherein Rᵥ is O or S & R_{λ} and R_{y} are the same as defined earlier) or NHC(═Rᵥ)NR_{λ}R_{y} (wherein Rᵥ, R_{y} and R_{λ} are the same as defined earlier). Unless otherwise constrained by the definition, all amino substituents may optionally be further substituted by 1-3 substituents chosen from alkyl, aralkyl, cycloalkyl, aryl, heteroaryl, heterocyclyl, carboxy, —COOR_{ψ} (wherein R_{ψ} is the same as defined earlier), hydroxy, alkoxy, halogen, CF₃, cyano, —C(═Rᵥ)NR_{λ}R_{y} (wherein Rᵥ is the same as defined earlier), —O(C═O)NR_{λ}R_{y}, —OC(═Rᵥ)NR_{λ}R_{y} (wherein R_{λ}, R_{y} and Rᵥ are the same as defined earlier), —S(O)ₘR_{ψ} (wherein R_{ψ} and m are the same as defined above).

The term “halogen” refers to fluorine, chlorine, bromine or iodine.

The compounds of the present invention can be used for treating CNS diseases, AIDS, asthma, arthritis, bronchitis, chronic obstructive pulmonary disease, psoriasis, allergic rhinitis, shock, atopic dermatitis, Crohn's disease, adult respiratory distress syndrome (ARDS), eosinophilic granuloma, allergic conjunctivitis, osteoarthritis, ulcerative colitis and other inflammatory diseases.

In accordance with yet another aspect, there are provided processes for the preparation of the compounds as described herein.

### DETAILED DESCRIPTION OF THE INVENTION

The compounds described herein may be prepared by techniques well known in the art and familiar to the average synthetic organic chemist. In addition, the compounds of present invention may be prepared, for example, by the following reaction sequences as depicted in Scheme I.

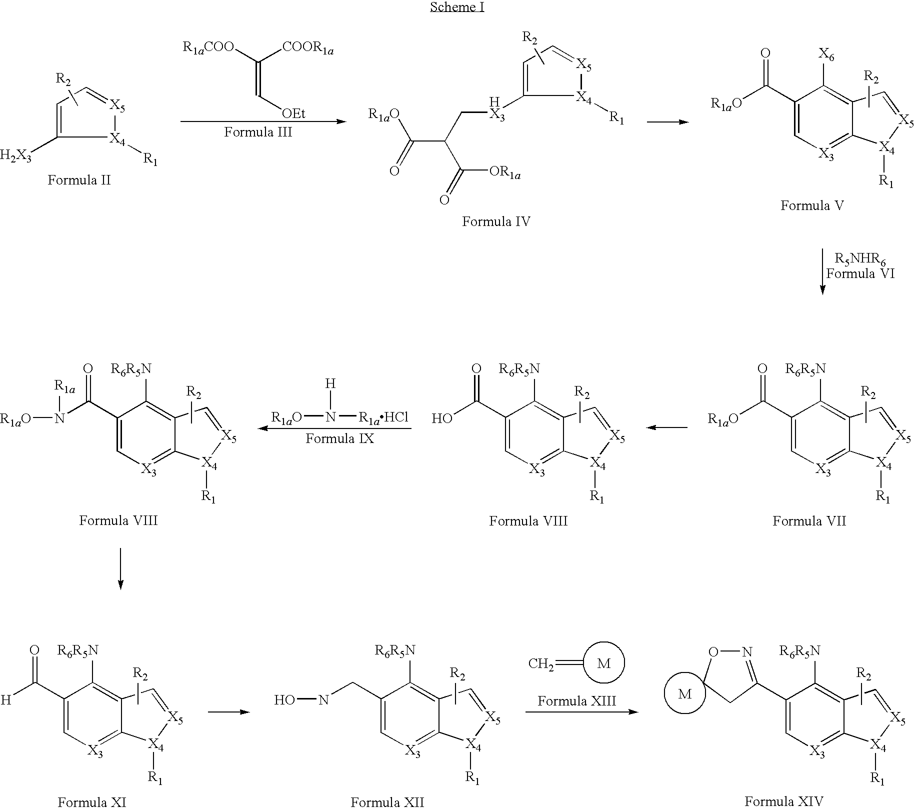

The compounds of Formula XIV can be prepared by following Scheme I. Thus, compounds of Formula II can be reacted with compounds of Formula III to give compounds of Formula IV (wherein R₁ₐ is alkyl), which on reaction with phosphorous oxy halide can give compounds of Formula V (wherein X₆ is a halogen), which on reaction with compounds of Formula VI can give compounds of Formula VII (wherein R₅ and R₆ are the same as defined earlier), which on ester hydrolysis can give compounds of Formula VIII, which on reaction with compounds of Formula IX can give compounds of Formula X, which on reduction can give compounds of Formula XI, which on reaction with hydroxylamine hydrochloride give compounds of Formula XII, which can be finally reacted with compounds of Formula XIII to give compounds of Formula XIV (wherein R₁, R₂, X₃, X₄, X₅ and M are the same as defined earlier).

The compounds of Formula IV can be prepared by the reaction of compounds of Formula II with compounds of Formula III with heating.

The compounds of Formula V can be prepared by the reaction of compounds of Formula IV with phosphorous oxy halide with heating.

The reaction of compounds of Formula V with compounds of Formula VI to give compounds of Formula VII can be carried out in one or more of nitrites, for example, acetonitrile, ketones, for example, acetone, alcohols, for example, ethanol, ethers, for example, tetrahydrofuran, amides, for example, dimethylformamide, sulfoxides, for example, dimethylsulfoxide or hydrocarbons, for example, toluene.

The ester hydrolysis of compounds of Formula VII to give compounds of Formula VIII can be carried out in one or more of alcohols, for example, methanol, ethanol or an alcohol and water mixture. The ester hydrolysis of compounds of Formula VII to give compounds of Formula VIII can be carried out in the presence of one or more of inorganic bases, for example, potassium hydroxide, sodium hydroxide or lithium hydroxide.

The reaction of compounds of Formula VIII with compounds of Formula IX to give compounds of Formula X can be carried out in the presence of one or more of activating reagents, for example, 1-hydroxybenzotriazole, acetone oxime or 2-hydroxypyridine, and one or more of coupling reagents, for example, 1-(3-dimethylaminopropyl)-3-ethyl carbodiimide hydrochloride or 1,3-dicyclohexyl carbodiimide in one or more of ethers, for example, tetrahydrofuran, amides, for example, dimethylformamide, sulfoxides, for example, dimethylsulfoxide.

The reaction of compounds of Formula VIII with compounds of Formula IX can be carried out in the presence of one or more of tertiary amine bases, for example, N-methylmorpholine, N-ethyldiisopropylamine or 4-dialkylaminopyridines.

The reduction of compounds of Formula X to give compounds of Formula XI can be carried out in one or more of ethers, for example, tetrahydrofuran, amides, for example, dimethylformamide, sulfoxides, for example, dimethylsulfoxide or hydrocarbons, for example, toluene.

The reduction of compounds of Formula X to give compounds of Formula XI can be carried out in the presence of one or more of reducing agents, for example, sodium bis(2-methoxyethoxy)aluminum hydride or lithium aluminium hydride.

The reaction of compounds of Formula XI with hydroxylamine hydrochloride to give compounds of Formula XII can be carried out in the presence of sodium acetate in one or more of alcohols, for example, methanol or ethanol.

The reaction of compounds of Formula XII with compounds of Formula XIII to give compounds of Formula XIV can be carried out in the presence of one or more halogenating agents, for example, sodium hypochlorite, N-chlorosuccinimide or N-bromosuccinimide in one or more of nitriles, for example, acetonitrile, ketones, for example, acetone, alcohols, for example, ethanol, ethers, for example, tetrahydrofuran, amides, for example, dimethylformamide, sulfoxides, for example, dimethylsulfoxide or hydrocarbons, for example, toluene.

In the above schemes, where the specific solvents, bases, reducing agents, oxidizing agents, activating reagents, coupling reagents, halogenating agents etc., are mentioned, it is to be understood that other solvents, bases, reducing agents, oxidizing agents, activating reagents, coupling reagents, halogenating agents etc., known to those skilled in the art may be used. Similarly, the reaction temperature and duration may be adjusted according to the desired needs.

An illustrative list of compounds includes these listed below:
N-cyclopropyl-1-ethyl-5-(1-oxa-2-azaspiro[4.5]dec-2-en-3-yl)-1H-pyrazolo[3,4-b]pyridin-4-amine (Compound No. 1),
N-cyclopropyl-1-ethyl-5-(1-oxa-2-azaspiro[4.4]non-2-en-3-yl)-1H-pyrazolo[3,4-b]pyridin-4-amine (Compound No. 2),
N-cyclopropyl-1-ethyl-5-(5-oxa-6-azaspiro[3.4]oct-6-en-7-yl)-1H-pyrazolo[3,4-b]pyridin-4-amine (Compound No. 3),
N-cyclopentyl-1,3-dimethyl-5-(5-oxa-6-azaspiro[3.4]oct-6-en-7-yl)-1H-pyrazolo[3,4-b]pyridin-4-amine (Compound No. 4),
N-cyclopentyl-1,3-dimethyl-5-(1-oxa-2-azaspiro[4.4]non-2-en-3-yl)-1H-pyrazolo[3,4-b]pyridin-4-amine (Compound No. 5),
N-cyclopentyl-1,3-dimethyl-5-(1-oxa-2-azaspiro[4.5]dec-2-en-3-yl)-1H-pyrazolo[3,4-b]pyridin-4-amine (Compound No. 6),
N-cyclopropyl-1,3-dimethyl-5-(1-oxa-2-azaspiro[4.4]non-2-en-3-yl)-1H-pyrazolo[3,4-b]pyridin-4-amine (Compound No. 7),
N-cyclopropyl-1,3-dimethyl-5-(5-oxa-6-azaspiro[3.4]oct-6-en-7-yl)-1H-pyrazolo[3,4-b]pyridin-4-amine (Compound No. 8),
N-cyclopropyl-1,3-dimethyl-5-(1-oxa-2-azaspiro[4.5]dec-2-en-3-yl)-1H-pyrazolo[3,4-b]pyridin-4-amine (Compound No. 9),
N-cyclopentyl-1-ethyl-5-(5-oxa-6-azaspiro[3.4]oct-6-en-7-yl)-1H-pyrazolo[3,4-b]pyridin-4-amine (Compound No. 10),
N-cyclopentyl-1-ethyl-5-(1-oxa-2-azaspiro[4.4]non-2-en-3-yl)-1H-pyrazolo[3,4-b]pyridin-4-amine (Compound No. 11), and
N-cyclopentyl-5-(1,7-dioxa-2-azaspiro[4.4]non-2-en-3-yl)-1,3-dimethyl-1H-pyrazolo[3,4-b]pyridin-4-amine (Compound No. 12),
   and their pharmaceutically acceptable salts, pharmaceutically acceptable solvates, stereoisomers, tautomers, racemates, regioisomers, prodrugs, metabolites, polymorphs or N-oxides, thereof.

The term “pharmaceutically acceptable” means approved by regulatory agency of the federal or a state government or listed in the U.S. Pharmacopoeia or other generally recognized pharmacopoeia for use in animals, and more particularly in humans.

The term “pharmaceutically acceptable salts” refers to derivatives of compounds that can be modified by forming their corresponding acid or base salts. Examples of pharmaceutically acceptable salts include, but are not limited to, mineral or organic acids salts of basic residues (such as amines), or alkali or organic salts of acidic residues (such as carboxylic acids), and the like.

The salt forms differ from the compound described herein in certain physical properties such as solubility, but the salts are otherwise equivalent for purposes of this invention.

The term “pharmaceutically acceptable solvates” refers to solvates with water (i.e. hydrates, hemihydrate or sesquihydrate) or pharmaceutically acceptable solvents, for example solvates with common organic solvents as ethanol and the like. Such solvates are also encompassed within the scope of the disclosure.

The present invention also includes within its scope prodrugs of these agents. In general, such prodrugs will be functional derivatives of these compounds, which are readily convertible in vivo into the required compound. Conventional procedures for the selection and preparation of prodrugs are known.

The disclosed compounds may get metabolized in vivo and these metabolites are also encompassed within the scope of this invention.

The term “polymorphs” includes all crystalline form as well as amorphous form for compounds described herein and as such are intended to be included in the present invention.

All stereoisomers of the compounds of the invention are contemplated, either in admixture or in pure or substantially pure form. The compounds of the present invention can have asymmetric centers at any of the carbon atoms including all the substituents. Consequently, compounds of present invention can exist in enantiomeric or diastereomeric forms or in mixture thereof. The processes for the preparation can utilize racemates, enantiomers, or diastereomers as starting materials. When diastereomeric or enantiomeric products are prepared, they can be separated by conventional methods, for example, chromatographic or fractional crystallization.

The term “tautomer” includes one of two or more structural isomers that exist in equilibrium and are readily converted from one isomeric form to another. Certain compounds of the general Formula (I) may furthermore be present in tautomeric forms.

The term “regioisomers” refers to compounds, which have the same molecular formula but differ in the connectivity of the atoms.

The term “compounds of the invention”, and equivalent expressions, are meant to embrace compounds of Formula (I) as herein described, including pharmaceutically acceptable salts, pharmaceutically acceptable solvates, stereoisomers, tautomers, racemates, regioisomers, prodrugs, metabolites, polymorphs or N-oxides, thereof, where the context so permits. In general and preferably, the compounds of the invention and the formulas designating the compounds of the invention are understood to only include the stable compounds thereof and exclude unstable compounds, even if an unstable compound might be considered to be literally embraced by the compound formula. Similarly, reference to intermediates, whether or not they themselves are claimed, is meant to embrace their salts and solvates, where the context so permits.

The term “stable compound” means a compound that is sufficiently robust to survive isolation to a useful degree of purity from a reaction mixture, and formulation into an efficacious therapeutic or diagnostic agent. For example, a compound, which would have a “dangling valency” or is a “carbanion” is not a compound contemplated by the invention.

The term “racemate” includes a mixture of equal amounts of left- and right-handed stereoisomers of chiral molecules.

When a bond to a substituent is shown to cross a bond connecting two atoms in a ring, then such substituent may be bonded to any atom on the ring.

The present disclosure includes all isotopes of atoms occurring in the present compounds. Isotopes include those atoms having the same atomic number but different mass numbers. By way of general example, and without limitation, isotopes of hydrogen include tritium and deuterium. Isotopes of carbon include ¹³C and ¹⁴C.

In another aspect, the present invention includes within its scope pharmaceutical compositions comprising, as an active ingredient, at least one of the disclosed compound or a pharmaceutically acceptable salt together with a pharmaceutically acceptable carrier or diluent. Compounds disclosed herein may be administered to human or animal for treatment by any route, which effectively transports the active compound to the appropriate or desired site of action such as oral, nasal, pulmonary, transdermal or parenteral (rectal, subcutaneous, intravenous, intraurethral, intramuscular, intranasal). The pharmaceutical composition of the present invention comprises a pharmaceutically effective amount of a compound of the present invention formulated together with one or more pharmaceutically acceptable carriers. The term “pharmaceutically acceptable carriers” is intended to include non-toxic, inert solid, semi-solid or liquid filler, diluents, encapsulating material or formulation of any type.

Where desired, the compounds of Formula I and/or their pharmaceutically acceptable salts, pharmaceutically acceptable solvates, stereoisomers, tautomers, racemates, regioisomers, prodrugs, metabolites, polymorphs or N-oxides may be advantageously used in combination with one or more other therapeutic agents. Examples of other therapeutic agents, which may be used in combination with compounds of Formula I of this invention and/or their pharmaceutically acceptable salts, pharmaceutically acceptable solvates, stereoisomers, tautomers, racemates, regioisomers, prodrugs, metabolites, polymorphs or N-oxides include corticosteroids, beta agonists, leukotriene antagonists, 5-lipoxygenase inhibitors, chemokine inhibitors and muscarinic receptor antagonists.

Examples set forth below demonstrate the synthetic procedures for the preparation of the representative compounds. The examples are provided to illustrate particular aspect of the disclosure and do not constrain the scope of the present invention as defined by the claims.

### EXPERIMENTAL DETAILS

### Example 1

### Preparation of Diethyl {[(1-ethyl-1H-pyrazol-5-yl)amino]methylene}malonate

A mixture of 5-amino-1-ethylpyrazole (1 gm, 0.0089 mole) and diethylethoxymethylenemalonate (1.9 ml, 0.0089 mole) was stirred at 120° C. for about 1 hour. The reaction mixture was poured into water and extraction was done with ethyl acetate. The organic layer was dried over anhydrous sodium sulfate and concentrated in vacuo to give yellow oil. Yield: 2.5 gm. m/z: (M⁺+1) 282.0.

The following compound was prepared similarly:
Diethyl {[(1,3-dimethyl-1H-pyrazol-5-yl)amino]methylene}malonate

### Example 2

### Preparation of ethyl-4-chloro-1-ethyl-1H-pyrazolo[3,4-b]pyridine-5-carboxylate

A mixture of diethyl {[(1-ethyl-1H-pyrazol-5-yl)amino]methylene}malonate (2.5 gm, 0.009 mole) and phosphorous oxy chloride (17.7 ml, 0.185 mole) was heated at 110-120° C. under stirring for about 3 hours under argon atmosphere. The reaction mixture was poured into ice water. It was extracted with dichloromethane and washed with brine. The organic layer was dried over anhydrous sodium sulfate and concentrated in vacuo to give pure white solid compound. Yield: 1.8 gm. m/z: (M⁺+1) 253.9.

The following compound was prepared similarly:
Ethyl-4-chloro-1,3-dimethyl-1H-pyrazolo[3,4-b]pyridine-5-carboxylate

### Example 3

### Preparation of ethyl-4-cyclopropylamino-1-ethyl-1H-pyrazolo[3,4-b]pyridine-5-carboxylate

To a mixture of ethyl 4-chloro-1-ethyl-1H-pyrazolo[3,4-b]pyridine-5-carboxylate (950 mg, 0.0037 mole) in acetonitrile, cyclopropyl amine (0.525 ml, 0.0074 mole) was added. After stirring for about 2 hours at 110° C., acetonitrile was removed under reduced pressure. Water was added and the reaction mixture was extracted with ethyl acetate. The organic layer was washed with brine, dried over anhydrous sodium sulfate and concentrated in vacuo to give light yellow solid compound. Yield: 1 gm. m/z: (M⁺+1) 275.0.

The following compounds were prepared similarly:
Ethyl 4-(cyclopropylamino)-1,3-dimethyl-1H-pyrazolo[3,4-b]pyridine-5-carboxylate
Ethyl 4-(cyclopentylamino)-1-ethyl-1H-pyrazolo[3,4-b]pyridine-5-carboxylate
Ethyl 4-(cyclopentylamino)-1,3-dimethyl-1H-pyrazolo[3,4-b]pyridine-5-carboxylate

### Example 4

### Preparation of 4-cyclopropylamino-1-ethyl-1H-pyrazolo[3,4-b]pyridine-5-carboxylic acid

To a solution of ethyl 4-cyclopropylamino-1-ethyl-1H-pyrazolo[3,4-b]pyridine-5-carboxylate (1 gm, 0.0036 mole) in ethanol, sodium hydroxide solution (440 mg in 2 ml water) was added. The reaction mixture was stirred for about 14 hours at ambient temperature. Water was added and the reaction mixture was extracted with ethyl acetate. Aqueous layer was acidified by using hydrochloric acid (2N) to pH of about 4-5. White solid, which was obtained, was filtered and dried in vacuo. Yield: 560 mg. m/z: (M⁺+1) 274.2.

The following compounds were prepared similarly:
4-(cyclopropylamino)-1,3-dimethyl-1H-pyrazolo[3,4-b]pyridine-5-carboxylic acid
4-(cyclopentylamino)-1-ethyl-1H-pyrazolo[3,4-b]pyridine-5-carboxylic acid
4-(cyclopentylamino)-1,3-dimethyl-1H-pyrazolo[3,4-b]pyridine-5-carboxylic acid

### Example 5

### Preparation of 4-(cyclopropylamino)-1-ethyl-N-methoxy-N-methyl-1H-pyrazolo[3,4-b]pyridine-5-carboxamide

4-cyclopropylamino-1-ethyl-1H-pyrazolo[3,4-b]pyridine-5-carboxylic acid (500 mg, 0.0020 mole) and N,O-dimethylhydroxylamine hydrochloride (298 mg, 0.0030 mole) were taken in dimethylformamide. At 0° C., hydroxybenzotriazole (550 mg, 0.0040 mole) and N-methylmorpholine (1.34 ml, 0.012 mole) were added and the reaction mixture was stirred for about 1 hour. 1-(3-dimethylaminopropyl) 3-ethyl carbodiimide hydrochloride (780 mg, 0.0040 mole) was added and the reaction mixture was stirred for about 14 hours. Water was added and extraction was carried out with ethyl acetate. The organic layer was washed with brine, dried over anhydrous sodium sulfate and concentrated in vacuo to give oily compound. Yield: 500 mg. m/z: (M⁺+1) 290.2.

The following compounds were prepared similarly:
4-(cyclopropylamino)-N-methoxy-N-1,3-trimethyl-1H-pyrazolo[3,4-b]pyridine-5-carboxamide
4-(cyclopentylamino)-1-ethyl-N-methoxy-N-methyl-1H-pyrazolo[3,4-b]pyridine-5-carboxamide
4-(cyclopentylamino)-N-methoxy-N-1,3-trimethyl-1H-pyrazolo[3,4-b]pyridine-5-carboxamide

### Example 6

### Preparation of 4-cyclopropylamino-1-ethyl-1H-pyrazolo[3,4-b]pyridine-5-carbaldehyde

Toluene was cooled at −19 to −20° C. and vitride (0.5 ml, 0.0034 mole) was added. After about 10 min., 4-(cyclopropylamino)-1-ethyl-N-methoxy-N-methyl-1H-pyrazolo[3,4-b]pyridine-5-carboxamide (500 mg, 0.0017 mole) was added and the reaction mixture was stirred for about 4 hours. Citric acid (10%) solution was added dropwise to quench the reaction and the reaction mixture was extracted with ethyl acetate. The organic layer was washed with brine and dried over anhydrous sodium sulfate and concentrated in vacuo to give oily compound. Yield: 300 mg. m/z: (M⁺+1) 231.1.

The following compounds were prepared similarly:
4-(cyclopropylamino)-1,3-dimethyl-1H-pyrazolo[3,4-b]pyridine-5-carbaldehyde
4-(cyclopentylamino)-1-ethyl-1H-pyrazolo[3,4-b]pyridine-5-carbaldehyde
4-(cyclopentylamino)-1,3-dimethyl-1H-pyrazolo[3,4-b]pyridine-5-carbaldehyde

### Example 7

### Preparation of 4-cyclopropylamino-1-ethyl-1H-pyrazolo[3,4-b]pyridine-5-carbaldehyde oxime

To a stirred solution of 4-cyclopropylamino-1-ethyl-1H-pyrazolo[3,4-b]pyridine-5-carbaldehyde (300 mg, 0.00129 mole) in ethanol, hydroxylamine hydrochloride (359 mg, 0.0051 mole) and sodium acetate (424 mg, 0.0051 mole) were added. The reaction mixture was allowed to stir at room temperature for about 50 minutes. Ethanol was removed under reduced pressure and residue was poured in water. The organic compound was extracted with ethyl acetate. Ethyl acetate layer was dried over anhydrous sodium sulfate, filtered and finally concentrated under reduced pressure to afford white solid compound. Yield: 270 mg. m/z: (M⁺+1) 246.1.

The following compounds were prepared similarly:
4-(cyclopropylamino)-1,3-dimethyl-1H-pyrazolo[3,4-b]pyridine-5-carbaldehyde oxime
4-(cyclopentylamino)-1-ethyl-1H-pyrazolo[3,4-b]pyridine-5-carbaldehyde oxime
4-(cyclopentylamino)-1,3-dimethyl-1H-pyrazolo[3,4-b]pyridine-5-carbaldehyde oxime

### Example 8

### Preparation of N-cyclopropyl-1-ethyl-5-(1-oxa-2-azaspiro[4.5]dec-2-en-3-yl)-1H-pyrazolo[3,4-b]pyridin-4-amine (Compound No. 1)

Methylene cyclohexane (0.029 ml, 0.0026 mole) was added to 4-cyclopropylamino-1-ethyl-1H-pyrazolo[3,4-b]pyridine-5-carbaldehyde oxime (40 mg, 0.00016 mol) in tetrahydrofuran. The reaction mixture was stirred at room temperature. Sodium hypochlorite (2 ml) was added slowly to the mixture thus obtained over a period of about 5 minutes and the reaction mixture was allowed to stir at room temperature for about 4 hours. Tetrahydrofuran was evaporated and the organic layer was extracted with ethyl acetate. It was concentrated and purified by column chromatography to yield the title compound. Yield: 15 mg (21.68%). m/z: (M⁺+1) 340.2.

The following compounds were prepared similarly:
N-cyclopropyl-1-ethyl-5-(1-oxa-2-azaspiro[4.4]non-2-en-3-yl)-1H-pyrazolo[3,4-b]pyridin-4-amine (Compound No. 2) Yield: (28.6%). m/z: (M⁺+1) 326.2.
N-cyclopropyl-1-ethyl-5-(5-oxa-6-azaspiro[3.4]oct-6-en-7-yl)-1H-pyrazolo[3,4-b]pyridin-4-amine (Compound No. 3) Yield: (15.87%). m/z: (M⁺+1) 312.2.
N-cyclopentyl-1,3-dimethyl-5-(5-oxa-6-azaspiro[3.4]oct-6-en-7-yl)-1H-pyrazolo[3,4-b]pyridin-4-amine (Compound No. 4) Yield: (28.6%). m/z: (M⁺+1) 340.1.
N-cyclopentyl-1,3-dimethyl-5-(1-oxa-2-azaspiro[4.4]non-2-en-3-yl)-1H-pyrazolo[3,4-b]pyridin-4-amine (Compound No. 5) Yield: (22%). m/z: (M⁺+1) 354.2.
N-cyclopentyl-1,3-dimethyl-5-(1-oxa-2-azaspiro[4.5]dec-2-en-3-yl)-1H-pyrazolo[3,4-b]pyridin-4-amine (Compound No. 6) Yield: (23.29%). m/z: (M⁺+1) 368.1.
N-cyclopropyl-1,3-dimethyl-5-(1-oxa-2-azaspiro[4.4]non-2-en-3-yl)-1H-pyrazolo[3,4-b]pyridin-4-amine (Compound No. 7) Yield: (23.44%). m/z: (M⁺+1) 326.1.
N-cyclopropyl-1,3-dimethyl-5-(5-oxa-6-azaspiro[3.4]oct-6-en-7-yl)-1H-pyrazolo[3,4-b]pyridin-4-amine (Compound No. 8) Yield: (15.74%). m/z: (M⁺+1) 312.1.
N-cyclopropyl-1,3-dimethyl-5-(1-oxa-2-azaspiro[4.5]dec-2-en-3-yl)-1H-pyrazolo[3,4-b]pyridin-4-amine (Compound No. 9) Yield: (18.11%). m/z: (M⁺+1) 340.1.
N-cyclopentyl-1-ethyl-5-(5-oxa-6-azaspiro[3.4]oct-6-en-7-yl)-1H-pyrazolo[3,4-b]pyridin-4-amine (Compound No. 10) Yield: (32.3%). m/z: (M⁺+1) 340.1.
N-cyclopentyl-1-ethyl-5-(1-oxa-2-azaspiro[4.4]non-2-en-3-yl)-1H-pyrazolo[3,4-b]pyridin-4-amine (Compound No. 11) Yield: (31%). m/z: (M⁺+1) 354.2.
N-cyclopentyl-5-(1,7-dioxa-2-azaspiro[4.4]non-2-en-3-yl)-1,3-dimethyl-1H-pyrazolo[3,4-b]pyridin-4-amine (Compound No. 12) Yield: (26%). m/z: (M⁺+1). 356.1.

### Example 9

### Efficacy of Compounds as PDE IV Inhibitors PDE-IV Enzyme Assay

The efficacy of compounds of PDE-4 inhibitors was determined by an enzyme assay using U937 cell cytosolic fraction (Biochem. Biophys. Res. Comm., 197: 1126-1131, (1993)). The enzyme reaction was carried out in the presence of cAMP (1 μM) at 30° C. in the presence or absence of test compound for 45-60 minutes. An aliquot of this reaction mixture was taken further for the ELISA assay and the protocol of the kit followed to determine level of cAMP in the sample. The concentration of the cAMP in the sample directly correlated with the degree of PDE-4 enzyme inhibition. Results were expressed as percent control and the IC₅₀ values of compounds were specifically disclosed herein found to be in the range of lower μM to nM concentration.

For example, K₃₀ values for the PDE IV assay ranged from about 0.15 μM to about 10 μM, or from about 0.15 μM to about 400 μM, or from about 0.15 μM to about 50 nM, or from about 0.15 μM to about 25 nM, or from about 0.15 μM to about 4 nM.

We claim:

1. Compounds having the structure of Formula I: 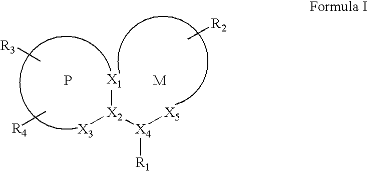
and their pharmaceutically acceptable salts, pharmaceutically acceptable solvates, stereoisomers, tautomers, racemates, regioisomers, prodrugs, metabolites, polymorphs or N-oxides, wherein ring P including X₁, X₂ and X₃ is a six membered ring containing 1-3 double bonds wherein X₁ and X₂ are carbon and X₃ is nitrogen;
ring M including X₁, X₂, X₄ and X₅ is a five membered ring containing 1-2 double bonds wherein X₁ and X₂ are carbon and X₄ and X₅ are nitrogen;
R₁ is hydrogen, alkyl, alkenyl, alkynyl, cycloalkyl, aryl, aralkyl, aralkenyl, (cycloalkyl) alkyl, heterocyclyl, heteroaryl, (heterocyclyl) alkyl or (heteroaryl) alkyl;
R₂ is hydrogen, alkyl, halogen, cyano, nitro, —SR, —NRR, —(CH₂)ₙ OR {wherein R is hydrogen, alkyl, alkenyl, alkynyl, cycloalkyl, aryl, heteroaryl or heterocyclyl and n is an integer from 0-2}, alkenyl, alkynyl, cycloalkyl, (cycloalkyl) alkyl, aryl, aralkyl, aralkenyl, heterocyclyl, heteroaryl, (heterocyclyl) alkyl or (heteroaryl) alkyl;
R₃ is —NR₅R₆ {wherein R₅ and R₆ independently are hydrogen, alkyl, alkenyl, alkynyl, acyl, cycloalkyl, aryl, aralkenyl, aralkyl, (cycloalkyl) alkyl, heterocyclyl, heteroaryl, (heterocyclyl) alkyl or (heteroaryl) alkyl};
R₄ is a radical of Formula I a or I b 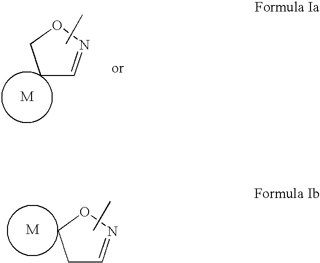
wherein M is a 3-7 membered saturated, partially saturated or unsaturated ring containing carbon atoms wherein one or more carbon atoms are replaced by heteroatoms selected from O, S(O)ₙ {wherein n is an integer from 0-2} or —NR-{wherein R is the same as defined earlier}.

2. Compounds, which are:
N-cyclopropyl-1-ethyl-5-(1-oxa-2-azaspiro[4.5]dec-2-en-3-yl)-1H-pyrazolo[3,4-b]pyridin-4-amine,
N-cyclopropyl-1-ethyl-5-(1-oxa-2-azaspiro[4.4]non-2-en-3-yl)-1H-pyrazolo[3,4-b]pyridin-4-amine,
N-cyclopropyl-1-ethyl-5-(5-oxa-6-azaspiro[3.4]oct-6-en-7-yl)-1H-pyrazolo[3,4-b]pyridin-4-amine,
N-cyclopentyl-1,3-dimethyl-5-(5-oxa-6-azaspiro[3.4]oct-6-en-7-yl)-1H-pyrazolo[3,4-b]pyridin-4-amine,
N-cyclopentyl-1,3-dimethyl-5-(1-oxa-2-azaspiro[4.4]non-2-en-3-yl)-1H-pyrazolo[3,4-b]pyridin-4-amine,
N-cyclopentyl-1,3-dimethyl-5-(1-oxa-2-azaspiro[4.5]dec-2-en-3-yl)-1H-pyrazolo[3,4-b]pyridin-4-amine,
N-cyclopropyl-1,3-dimethyl-5-(1-oxa-2-azaspiro[4.4]non-2-en-3-yl)-1H-pyrazolo[3,4-b]pyridin-4-amine,
N-cyclopropyl-1,3-dimethyl-5-(5-oxa-6-azaspiro[3.4]oct-6-en-7-yl)-1H-pyrazolo[3,4-b]pyridin-4-amine,
N-cyclopropyl-1,3-dimethyl-5-(1-oxa-2-azaspiro[4.5]dec-2-en-3-yl)-1H-pyrazolo[3,4-b]pyridin-4-amine,
N-cyclopentyl-1-ethyl-5-(5-oxa-6-azaspiro[3.4]oct-6-en-7-yl)-1H-pyrazolo[3,4-b]pyridin-4-amine,
N-cyclopentyl-1-ethyl-5-(1-oxa-2-azaspiro[4.4]non-2-en-3-yl)-1H-pyrazolo[3,4-b]pyridin-4-amine,
N-cyclopentyl-5-(1,7-dioxa-2-azaspiro[4.4]non-2-en-3-yl)-1,3-dimethyl-1H-pyrazolo[3,4-b]pyridin-4-amine,
and their pharmaceutically acceptable salts, pharmaceutically acceptable solvates, stereoisomers, tautomers, racemates, regioisomers, prodrugs, metabolites, polymorphs or N-oxides.

3. Pharmaceutical compositions comprising a therapeutically effective amount of a compound of claim 1, together with at least one pharmaceutically acceptable carrier, excipient or diluent.

4. Pharmaceutical compositions comprising a therapeutically effective amount of a compound of claim 1 and at least one other active ingredient selected from corticosteroids, beta agonists, leukotriene antagonists, 5-lipoxygenase inhibitors, chemokine inhibitors and muscarinic receptor antagonists.

5. A method for treating, preventing, inhibiting or suppressing an inflammatory condition or disease or CNS diseases in a patient, comprising administering to the patient a therapeutically effective amount of a compound of claim 1.

6. A method for treating, preventing, inhibiting or suppressing an inflammatory condition or disease or CNS diseases in a patient, comprising administering to the patient a therapeutically effective amount of a pharmaceutical composition of claim 3.

7. A method for the treatment, prevention, inhibition or suppression of CNS diseases, AIDS, asthma, arthritis, bronchitis, chronic obstructive pulmonary disease (COPD), psoriasis, allergic rhinitis, shock, atopic dermatitis, Crohn's disease, adult respiratory distress syndrome (ARDS), eosinophilic granuloma, allergic conjunctivitis, osteoarthritis, ulcerative colitis and other inflammatory diseases in a patient comprising administering to the patient a therapeutically effective amount of a compound of claim 1.

8. A method for the treatment, prevention, inhibition or suppression of CNS diseases, AIDS, asthma, arthritis, bronchitis, chronic obstructive pulmonary disease (COPD), psoriasis, allergic rhinitis, shock, atopic dermatitis, Crohn's disease, adult respiratory distress syndrome (ARDS), eosinophilic granuloma, allergic conjunctivitis, osteoarthritis, ulcerative colitis and other inflammatory diseases in a patient comprising administering to the patient a therapeutically effective amount of a pharmaceutical composition of claim 3.

9. A method for the preparation of compounds of Formula XIV, 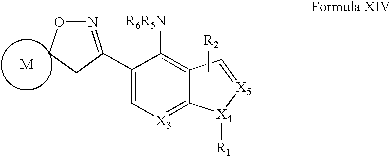
and their pharmaceutically acceptable salts, pharmaceutically acceptable solvates, stereoisomers, tautomers, racemates, regioisomers, prodrugs, metabolites, polymorphs or N-oxides, wherein
M is a 3-7 membered saturated, partially saturated or unsaturated ring containing carbon atoms wherein one or more carbon atoms are replaced by heteroatoms selected from O, S(O)ₙ {wherein n is an integer from 0-2} or —NR— {wherein R is hydrogen, alkyl, alkenyl, alkynyl, cycloalkyl, aryl, heteroaryl or heterocyclyl and n is an integer from 0-2};
R₁ is hydrogen, alkyl, alkenyl, alkynyl, cycloalkyl, aryl, aralkyl, aralkenyl, (cycloalkyl) alkyl, heterocyclyl, heteroaryl, (heterocyclyl) alkyl or (heteroaryl) alkyl;
R₂ is hydrogen, alkyl, halogen, cyano, nitro, —SR, —NRR, —(CH₂)ₙ OR {wherein R is hydrogen, alkyl, alkenyl, alkynyl, cycloalkyl, aryl, heteroaryl or heterocyclyl and n is an integer from 0-2}, alkenyl, alkynyl, cycloalkyl, (cycloalkyl) alkyl, aryl, aralkyl, aralkenyl, heterocyclyl, heteroaryl, (heterocyclyl) alkyl or (heteroaryl) alkyl;
R₅ and R₆ independently are hydrogen, alkyl, alkenyl, alkynyl, acyl, cycloalkyl, aryl, aralkenyl, aralkyl, (cycloalkyl) alkyl, heterocyclyl, heteroaryl, (heterocyclyl) alkyl or (heteroaryl) alkyl}; and
X₃, X₄ and X₅ are nitrogen,
the method comprising
(a) reacting a compound of Formula II with a compound of Formula III to give a compound of Formula IV (wherein R₁ₐ is independently alkyl); 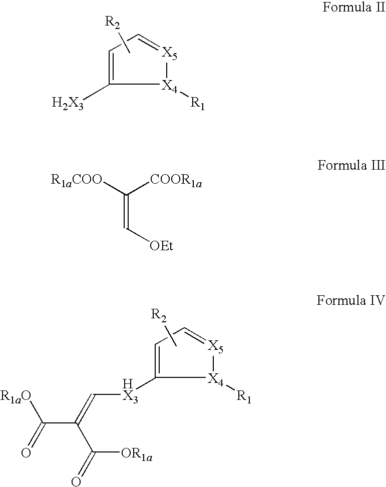
(b) reacting the compound of Formula IV with phosphorous oxy halide to give a compound of Formula V (wherein X₆ is a halogen); 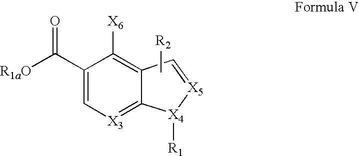
(c) reacting the compound of Formula V with a compound of Formula VI to give a compound of Formula VII (wherein R₅ and R₆ are the same as defined earlier); 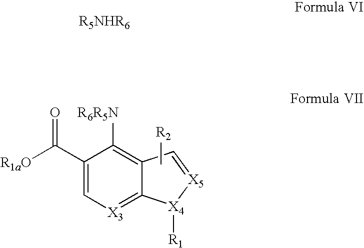
(d) performing ester hydrolysis of the compound of Formula VII to give a compound of Formula VIII; 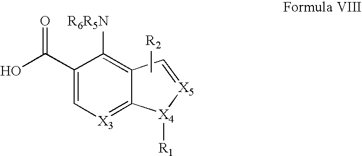
(e) reacting the compound of Formula VIII with a compound of Formula IX to give a compound of Formula X; 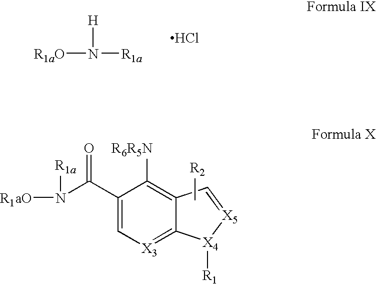
(f) reduction of the compound of Formula X to give compound of Formula XI; 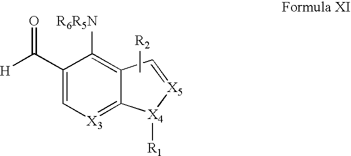
(g) reacting the compound of Formula XI with hydroxylamine hydrochloride to give a compound of Formula XII; and 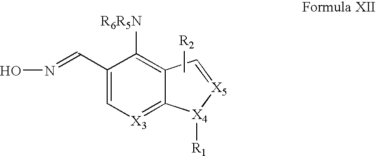
(h) reacting the compound of Formula XII with a compound of Formula XIII (where M is as defined above) 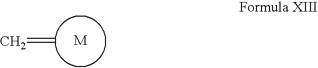
to give the compound of Formula XIV.
